(19) United States
(12) Patent Application Publication
(10) Pub. No.: US 2007/0112280 A1
(43) Pub. Date: May 17, 2007

(54) Placental-blood extraction device

(75) Inventor: Patrice Richard, Paris (FR)

Correspondence Address: SUGHRUE MION, PLLC, 2100 PENNSYLVANIA AVENUE, N.W., SUITE 800, WASHINGTON, DC, 20037, US

(73) Assignee: PRAXCELL, Toulouse (FR)

(21) Appl. No.: 10/577,132

(22) PCT Filed: Oct. 27, 2004
(86) PCT No.: PCT/FR04/50542

§ 371 Date: Aug. 28, 2006

(30) Foreign Application Priority Data
Oct. 27, 2003 (FR) 03 12543

(51) Int. Cl. A61B 5/00; A61B 19/00

(52) U.S. Cl. 600/573; 604/403; 604/416

(57) ABSTRACT

A placental-blood extraction device comprising at least one extraction needle (10, 11) for piercing the vein of the umbilical cord and/or of the placenta, and a collection vessel (20) connected to said at least one needle (10, 11) via at least one tube (40), the device further comprising suction means (30) connected to said at least one needle,(10, 11) and adapted to suck the placental blood so as to feed said collection vessel (20), said suction means (30) comprising a vacuum bottle (20), in particular of the Redon type, that simultaneously forms a collection vessel.

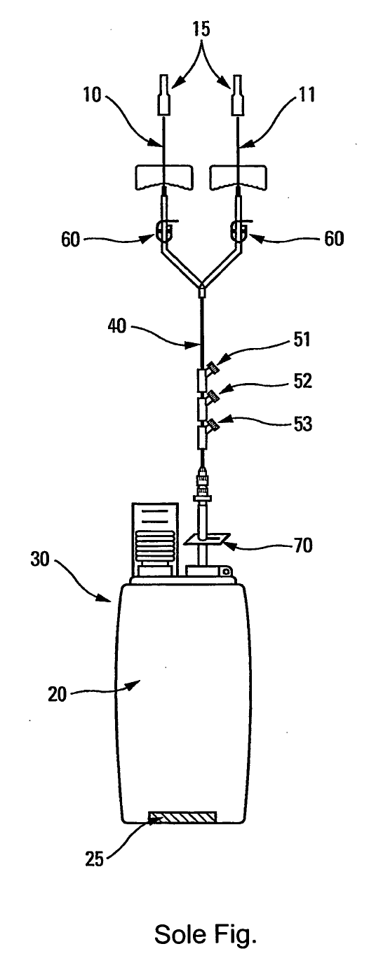

The present invention relates to a placental-blood extraction device.

During childbirth, it is known to extract placental blood, also known as umbilical-cord blood, which presents particularly advantageous properties. The blood is generally extracted after the baby has been separated from the umbilical cord. The person responsible for performing the extraction thus uses a system that includes one or more extraction needles for piercing the vein of the cord so as to extract the blood contained in the cord, by gravity. This operation is relatively lengthy, typically taking about twenty minutes, which, in view of the blood-coagulation process, consequently limits the quantities extracted and the quality of the extracted blood.

An object of the present invention is to provide a placental-blood extraction device that does not have the above-mentioned drawbacks.

More particularly, an object of the present invention is to provide a placental-blood extraction device that is more effective, and that makes it possible to optimize the quantity of blood extracted and the quality thereof.

Another object of the present invention is to provide such a device that is simple, inexpensive, and safe to manufacture and to use, in particular with regard to sterility.

The present invention thus provides a placental-blood extraction device comprising at least one extraction needle for piercing the vein of the umbilical cord and/or of the placenta, and a collection vessel connected to said at least one needle via at least one tube, the device further comprising suction means connected to said at least one needle and adapted to suck the placental blood so as to feed said collection vessel, said suction means comprising a vacuum bottle, in particular of the Redon type, that simultaneously forms a collection vessel.

Advantageously, said suction means further comprises a vacuum pump.

The device advantageously includes at least one injection and/or extraction site between said at least one extraction needle and said collection vessel.

At least one injection and/or extraction site is advantageously provided on the tube.

At least one injection and/or extraction site is advantageously provided on the collection vessel.

Advantageously, said at least one injection and/or extraction site is used to inject an anti-coagulant and/or to extract a sample of blood for analysis and/or to extract the blood contained in said collection vessel.

Advantageously, said device includes blood-flow control means and/or suction control means.

Advantageously, said blood-flow control means and/or said suction control means include a knurled adjustment wheel.

Advantageously, said collection vessel contains an anti-coagulant before receiving said placental blood.

Advantageously, said device is packaged in sterile manner and assembled in a single package so as to be ready to use once said package has been opened.

Other characteristics and advantages of the present invention appear more clearly from the following detailed description of an advantageous embodiment thereof, given by way of non-limiting example, and with reference to the sole figure which is a diagram showing a placental-blood extraction device constituting a particular embodiment of the present invention.

In known manner, the device includes at least one extraction needle 10, 11. Two needles are preferably provided, thereby enabling extraction to be performed at two different locations, and at two successive moments. Safety systems 15 are advantageously provided on each needle 10, 11 so as to avoid any risk of injury before and after extraction. The needle(s) 10, 11 is/are connected to a collection vessel 20 for receiving the extracted blood. A tube 40 advantageously connects said collection vessel 20 to said at least one needle 10, 11.

In the invention, the device includes suction means 30 for sucking the placental blood into the collection vessel 20. The suction means therefore promote rapid transfer into the collection vessel 20 of the blood to be extracted, thereby making it possible to extract a greater quantity of blood, to accelerate the extraction process, to improve the quality of the extracted blood, and to limit the risks of said extracted blood coagulating before it arrives in the collection vessel 20.

The embodiment shown in the figure shows the use of a Redon-type vacuum bottle which is particularly advantageous for the present invention. A Redon bottle is a vacuum vessel that is used after surgery in order to suck up the last of the post-operative liquid waste from the patient. The use of such a Redon bottle in the present invention makes it possible for the extraction to be fast and of high quality. The suction means can also include additional suction means, e.g. a vacuum pump, or a peristaltic pump, or any other type of pump. Naturally, other suction means could also be envisaged. For example, an extraction reservoir that is connected to, or disposed inside, a vacuum vessel could also be used, and other systems could also be used.

In an advantageous aspect of the present invention, the extraction device includes at least one injection and/or extraction site 51, 52, 53 provided between said at least one needle 10, 11 and said collection vessel. In the embodiment shown, there are three sites of this type, namely an injection site 51 for injecting an anti-coagulant, e.g. heparin or citrate phosphate dextrose (CPD), during the extraction process, a first extraction site 52 for extracting a sample of blood during the extraction process in order to analyze it, e.g. in order to carry out hepatitis or HIV tests, and a second extraction site 53 for extracting all of the blood contained in the collection vessel 20 at the end of the extraction process. The injection and/or extraction sites 51, 52, 53 can be disposed on the tube 40, as shown, but they could also advantageously be formed directly on the collection vessel 20. In addition, and in advantageous manner, it is possible to envisage disposing an appropriate quantity of anti-coagulant, e.g. heparin, in the collection vessel 20 before starting the extraction process. In a variant, it is possible to provide an isolated pouch containing an anti-coagulant and connected to the device, e.g. at said injection site. By way of example, the pouch can be isolated by a device that is adjustable or breakable, and that optionally enables the contents of said pouch to flow into the collection vessel, depending on requirements.

Advantageously, it is possible to provide a stirrer in the vessel 20, e.g. a magnetic or a movable stirrer. In particular, this makes it possible to improve the mixing of the anti-coagulant with the extracted blood.

In order to ensure that the device operates properly, the suction must not be too great, since that might risk causing a constriction or blockage in the tube 40, nor must it be too small in order to be effective. The device therefore advantageously includes suction control means 70 that can be made in the form of an element that is adapted to modify the section of the tube 40. A knurled adjustment wheel could advantageously be used. In a variant, control means that are provided directly on the suction means 30, or on the collection vessel 20, could also be envisaged. In addition, blood-flow control means 60 are advantageously provided, and are advantageously disposed directly downstream from the extraction needles 10, 11, with said blood-flow control means also being made in any desired way, e.g. by means of a knurled adjustment wheel.

Filters can be provided at the various extraction and/or injection sites, as well as upstream from the vessel 20. For example, the presence of such a filter can enable the vessel to be taken to atmospheric pressure as soon as extraction is complete, without risk of contamination and in sterile conditions.

A particular advantage of the present invention relates to its safety in use, and in particular to its sterile character. The device of the invention can be assembled, sterilized, and then packaged in sterile manner in a single package, such that when placental blood is to be extracted, the user has only to open the sterile package and pierce the umbilical cord in order to extract the blood, without having to perform any assembly operations. The sterility of the device is therefore guaranteed at all times.

The present invention therefore makes it possible to provide a placental-blood extraction device that simplifies the extraction process, that makes it safer, and that makes it more effective by optimizing both the quantity of blood extracted and the quality of the extracted blood.

Although the present invention is described above with reference to a particular embodiment thereof, naturally it is not limited by the embodiment shown. On the contrary, any useful modifications can be applied thereto by the person skilled in the art, without going beyond the ambit of the present invention, as defined by the accompanying claims.

1. A placental-blood extraction device comprising at least one extraction needle (10, 11) for piercing the vein of the umbilical cord and/or of the placenta, and a collection vessel (20) connected to said at least one needle (10, 11) via at least one tube (40), the device being characterized in that it further comprises suction means (30) connected to said at least one needle (10, 11) and adapted to suck the placental blood so as to feed said collection vessel (20), said suction means (30) comprising a vacuum bottle (20), in particular of the Redon type, that simultaneously forms a collection vessel.

2. A device according to claim 1, in which said suction means (30) further comprise a vacuum pump.

3. A device according to claim 1, in which the device includes at least one injection and/or extraction site (51, 52, 53) between said at least one extraction needle (10, 11) and said collection vessel (20).

4. A device according to claim 3, in which at least one injection and/or extraction site (51, 52, 53) is provided on the tube (40).

5. A device according to claim 3, in which at least one injection and/or extraction site (51, 52, 53) is provided on the collection vessel (20).

6. A device according to claim 3, in which said at least one injection and/or extraction site (51, 52, 53) is used to inject an anti-coagulant and/or to extract a sample of blood for analysis and/or to extract the blood contained in said collection vessel (20).

7. A device according to claim 1, in which said device includes blood-flow control means (60) and/or suction control means (70).

8. A device according to claim 7, in which said blood-flow control means (60) and/or said suction control means (70) include a knurled adjustment wheel.

9. A device according to claim 1, in which said collection vessel (20) contains an anti-coagulant before receiving said placental blood.

10. A device according to claim 1, in which said device is packaged in sterile manner and is assembled in a single package so as to be ready to use once said package has been opened.
